(19) United States
(12) Patent Application Publication
(10) Pub. No.: US 2005/0260249 A1
(43) Pub. Date: Nov. 24, 2005

(54) Antimicrobial contact lenses and methods for their production

(76) Inventors: Frank L. Neely, Jacksonville, FL (US); Azaam Alli, Jacksonville, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/748,621

(22) Filed: Dec. 30, 2003

Related U.S. Application Data
(63) Continuation-in-part of application No. 10/028,400, filed on Dec. 20, 2001.
(60) Provisional application No. 60/257,030, filed on Dec. 21, 2000.

(51) Int. Cl. A61K 33/38; A61F 2/00

(52) U.S. Cl. 424/427; 424/618

(57) ABSTRACT

This invention relates to antimicrobial lenses and methods for their production where the lenses contain silver and at least one ligand monomer of Formula I, 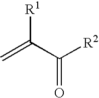
where R¹, R² are defined herein and the ratio of silver to is at least about 0.6.

### RELATED INVENTIONS

This patent application claims priority from U.S. Ser. No. 10/028,400, that was filed on Dec. 20, 2001, which claimed priority from provisional application U.S. Ser. No. 60/257,030, filed on Dec. 21, 2000.

### FIELD OF THE INVENTION

This invention relates to contact lenses having antimicrobial properties as well as methods of their production, use, and storage.

### BACKGROUND OF THE INVENTION

Contact lenses have been used commercially to improve vision since the 1950s. The first contact lenses were made of hard materials. Although these lenses are currently used, they are not suitable for all patients due to their poor initial comfort and their relatively low permeability to oxygen. Later developments in the field gave rise to soft contact lenses, based upon hydrogels, which are extremely popular today. Many users find soft lenses are more comfortable, and increased comfort levels allow soft contact lens users to wear their lenses for far longer hours than users of hard contact lenses.

Despite this advantage, the extended use of the lenses can encourage the buildup of bacteria or other microbes, particularly, Pseudomonas aeruginosa, on the surfaces of soft contact lenses. The build-up of bacteria or other microbes is not unique to soft contact lens wearers and may occur during the use of hard contact lenses as well.

Therefore, there is a need to produce contact lenses that inhibit the growth of bacteria or other microbes and/or the adhesion of bacterial or other microbes on the surface of contact lenses. Further there is a need to produce contact lenses which do not promote the adhesion and/or growth of bacteria or other microbes on the surface of the contact lenses. Also there is a need to produce contact lenses that inhibit adverse responses related to the growth of bacteria or other microbes.

Although methods and lenses are known, other contact lenses that inhibit the growth and/or adhesion of bacteria or other microbes and are of sufficient optical clarity, as well as methods of making those lenses are still needed. It is this need, which this invention seeks to meet.

### DETAILED DESCRIPTION OF THE INVENTION

This invention includes an antimicrobial lens having improved antimicrobial efficacy. Specifically, the lenses of the present invention have metal to ligand ratio of greater than about 0.6, and preferably 0.8.

The lenses of the present invention comprise, consist essentially of, or consist of, silver and a polymer comprising at least one ligand monomer of Formula I 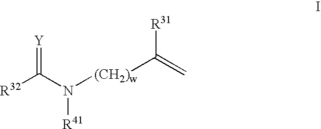
wherein
w is 0-1;
Y is oxygen or sulfur;
R³¹ is hydrogen or C₁₋₆alkyl;
R³² is hydroxyl, amino, sulfonic acid, phosphonic acid, carboxylic acid, thioC₁₋₆alkylcarbonyl, thioC₁₋₆alkylaminocarbonyl, —C(O)NH—(CH₂)_{d}—R³³, —O—R³³, —NH—R³³, —S—(CH₂)_{d}—R³³, —(CH₂)_{d}—R³³, C₁₋₆alkyldisulfide, phenyldisulfide, urea, C₁₋₆alkylurea, phenylurea, thiourea, C₁₋₆alkylthiourea, phenylthiourea, C₁₋₆alkylamine, phenylamine, substituted C₁₋₆alkyldisulfide, substituted phenyldisulfide, substituted phenylurea, substituted C₁₋₆alkylamine, substituted phenylamine, substituted phenylthiourea, substituted C₁₋₆alkylurea or substituted C₁₋₆alkylthiourea wherein the substitutents are selected from the group consisting of C₁₋₆alkyl, haloC₁₋₆alkyl, halogen, hydroxyl, carboxylic acid, sulfonic acid, phosphonic acid, amine, amidine, acetamide, and nitrile
   where
      d is 0-8;
      R³³ is thioC₁₋₆alkylcarbonyl, C₁₋₆alkyl, substituted C₁₋₆alkyl where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea or substituted phenylthiourea
         wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
      —(CR³⁴R³⁵)_{q}—(CHR³⁶)ₘ—SO₃H
         where R³⁴, R³⁵, and R³⁵ are independently selected from the group consisting of hydrogen, halogen, hydroxyl, and C₁₋₆alkyl,
         q is 1-6, and m is 0-6;
      —(CH₂)ₙ—S—S—(CH₂)ₙNH—C(O)CR³⁷CH₂,
         where R³⁷ is hydrogen or C₁₋₆alkyl,
         n is 1-6, and x is 1-6;
      —(CR³⁸R³⁹)ₜ—(CHR⁴⁰)ₙ—P(O)(OH)₂
         where R³⁸, R3⁹, and R⁴⁰ are independently selected from the group consisting of hydrogen, halogen, hydroxyl, and C₁₋₆alkyl, t is 1-6, and u is 0-6;
      phenyl, benzyl, pyridinyl, pyrimidinyl, pyrazinyl, benzimidazolyl, benzothiazolyl, benzotriazolyl, naphthaloyl, quinolinyl, indolyl, thiadiazolyl, triazolyl, 4-methylpiperidin-1-yl, 4-methylpiperazin-1-yl, substituted phenyl, substituted benzyl, substituted pyridinyl, substituted pyrimidinyl, substituted pyrazinyl, substituted benzimidazolyl, substituted benzothiazolyl, substituted benzotriazolyl, substituted naphthaloyl, substituted quinolinyl, substituted indolyl, substituted thiadiazolyl, substituted triazolyl, substituted 4-methylpiperidin-1-yl, or substituted 4-methylpiperazin-1-yl,
         wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl)phosphonyl, N-(amino-4-methylpiperazinyl)phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
         wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
R⁴¹ is hydrogen, C₁₋₆alkyl, phenyl, C₁₋₆alkylcarbonyl, phenylcarbonyl, substituted C₁₋₆alkyl, substituted phenyl, substituted C₁₋₆alkylcarbonyl or substituted phenylcarbonyl,
   wherein
      the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile.

The preferred ligand monomers include monomers where
w is 0-1;
R³¹ is hydrogen;
R³² is amine, C₁₋₃alkylamine, phenylamine, substituted phenylamine, thioC₁₋₃alkylcarbonyl;
R⁴¹ is hydrogen
   The more preferred ligand monomers include 1-allyl-2 thiourea and the following monomers

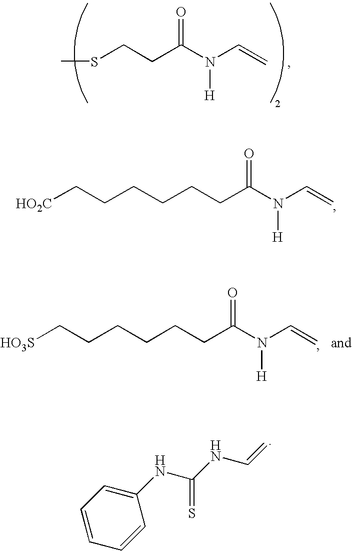

Mixtures of ligand monomers may also be used. In a particularly preferred embodiment the at least one ligand monomer comprises 1-allyl-2-thiourea.

As used herein, the term “lens” refers to opthalmic devices that reside in or on the eye. These devices can provide optical correction or may be cosmetic. The term lens includes but is not limited to soft contact lenses, hard contact lenses, intraocular lenses, overlay lenses, ocular inserts, and optical inserts. Soft contact lenses are made from silicone elastomers or hydrogels, which include but are not limited to silicone hydrogels and fluorohydrogels. These hydrogels may be formed from lens forming components, including hydrophobic and/or hydrophilic monomers that are covalently bound to one another in the cured lens.

As used herein the term “polymers” means copolymers, homopolymers, or mixtures thereof. The ligand monomers or their homopolymers, are added to the monomer mix of contact lenses, prior to polymerization in an amount based on the weight percent of the initial monomer mix, including a suitable diluent if said diluent is used in the preparation of the polymer. The weight percentage of the ligand monomers of the invention can vary with the lens formulation. The maximum percentage of ligand monomers is the percentage that does not compromise the physical properties of the resulting contact lens, such as, but not limited to modulus, of the resulting lens. The minimum percentage of ligand monomer is an amount that allows the incorporation of a sufficient amount of silver into a lens to provide the desired antimicrobial effect. Preferably, about 0.01 to about 20.0 weight percent of at least one ligand monomer is added, to a monomer mix, more preferably, about 0.01 to about 1.5 weight percent, even more preferably, about 0.01 to about 0.4 weight percent, most preferably, about 0.05 to about 0.2 weight percent, all based upon the total lens forming components in the monomer mix.

Suitable lens forming components are known in the art and include acrylic- or vinyl-containing monomers, hydrophobic monomers and macromers internal wetting agents and compatibilizing monomers and macromers, initiators, UV absorbing compounds, visibility tints, crosslinkers combinations thereof and the like. Acrylic-containing monomers contain the acrylic group: (CH₂═CRCOX—) wherein R is H or CH₃, and X is O or N, polymerize readily and include, but are not limited to N,N-dimethyl acrylamide (DMA), 2-hydroxyethyl methacrylate (HEMA), glycerol methacrylate, 2-hydroxyethyl methacrylamide, polyethyleneglycol monomethacrylate, methacrylic acid and acrylic acid.

Vinyl-containing monomers contain the vinyl grouping (—CH═CH₂), and include but are not limited to monomers such as N-vinyl lactams (such as, but not limited to N-vinylpyrrolidone, or NVP), N-vinyl-N-methyl acetamide, N-vinyl-N-ethyl acetamide, N-vinyl-N-ethyl formamide, N-vinyl formamide, with NVP being preferred.

As used herein the term “compatibilizing monomers and macromers” mean reaction components which contain at least one silicone group and at least one hydroxyl group. Such components have been disclosed in U.S. Pat. No. 6,367,929, WO03/022321 and WO03/022322, the disclosures of which are incorporated herein in their entirety, along with any other patents or applications which are referenced herein. A suitable example includes 3-methacryloxy-2-hydroxypropyloxypropylbis(trimethylsiloxy)methylsilane.

Suitable hydrophobic components include silicone containing components and fluorine containing components. Silicone-containing components contain at least one [—Si—O—Si] group, and at least one polymerizable functional group in a monomer, macromer or prepolymer. Preferably, the Si and attached 0 are present in the silicone-containing component in an amount greater than 20 weight percent, and more preferably greater than 30 weight percent of the total molecular weight of the silicone-containing component. Examples of silicone-containing components which are useful in this invention may be found in U.S. Pat. Nos. 3,808,178; 4,120,570; 4,136,250; 4,153,641; 4,740,533; 5,034,461, 5,070,215, WO03/022322, WO03/022321, U.S. Pat. No. 6,367,929, U.S. Pat. Nos. 5,998,498, 5,760,100, 5,260,000, 4,711,943, 4,139,513, U.S. Pat. No. 4,139,548, U.S. Pat. No. 4,235,985 and EP080539. Examples of suitable hydrophobic monomers include, but are not limited to tris(trimethylsiloxy)silylpropyl methacrylate, monomethacryloxypropyl terminated polydimethylsiloxanes, polydimethylsiloxanes, 3-methacryloxypropylbis(trimethylsiloxy)methylsilane, methacryloxypropylpentamethyl disiloxane, N-tris(trimethylsiloxy)-silylpropylmethacrylamide, N-tris(trimethylsiloxy)-silylpropylacrylamide and combinations thereof.

Silicone hydrogels of the present invention may also include an internal wetting agent, such as, but not limited to at least one “high molecular weight hydrophilic polymer”, which refers to substances having a weight average molecular weight of no less than about 100,000 Daltons, wherein said substances upon incorporation to silicone hydrogel formulations, increase the wettability of the cured silicone hydrogels. Suitable high molecular weight hydrophilic polymers are disclosed in WO03/022321, which is incorporated in its entirety herein by reference.

Suitable amounts of high molecular weight hydrophilic polymer include from about 1 to about 15 weight percent, more preferably about 3 to about 15 percent, most preferably about 3 to about 12 percent, all based upon the total of all lens forming components.

Examples of high molecular weight hydrophilic polymers include but are not limited to polyamides, polylactones, polyimides, polylactams and functionalized polyamides, polylactones, polyimides, polylactams. Hydrophilic prepolymers made from DMA or n-vinyl pyrrolidone with glycidyl methacrylate may also be used. The glycidyl methacrylate ring can be opened to give a diol which may be used in conjunction with other hydrophilic prepolymer in a mixed system to increase the compatibility of the high molecular weight hydrophilic polymer, hydroxyl-functionalized silicone containing monomer and any other groups which impart compatibility. The preferred high molecular weight hydrophilic polymers are those that contain a cyclic moiety in their backbone, more preferably, a cyclic amide or cyclic imide. High molecular weight hydrophilic polymers include but are not limited to poly-N-vinyl pyrrolidone, poly-N-vinyl-2-piperidone, poly-N-vinyl-2-caprolactam, poly-N-vinyl-3-methyl-2-caprolactam, poly-N-vinyl-3-methyl-2-piperidone, poly-N-vinyl-4-methyl-2-piperidone, poly-N-vinyl-4-methyl-2-caprolactam, poly-N-vinyl-3-ethyl-2-pyrrolidone, and poly-N-vinyl-4,5-dimethyl-2-pyrrolidone, polyvinylimidazole, poly-N-N-dimethylacrylamide, polyvinyl alcohol, polyacrylic acid, polyethylene oxide, poly 2 ethyl oxazoline, heparin polysaccharides, polysaccharides, mixtures and copolymers (including block or random, branched, multichain, comb-shaped or star shaped) thereof where poly-N-vinylpyrrolidone (PVP) is preferred.

Other lens forming components such as crosslinkers, UV absorbing agents, tinting agents are known in the art and need not be described here.

The type of initiator used in the present invention is not critical. Suitable intitiators include thermal initators such as lauryl peroxide, benzoyl peroxide, isopropyl percarbonate, azobisisobutyronitrile, and the like, that generate free radicals at moderately elevated temperatures, and photoinitiator systems such as aromatic alpha-hydroxy ketones, alkoxyoxybenzoins, acetophenones, acylphosphine oxides, bisacylphosphine oxides, and a tertiary amine plus a diketone, mixtures thereof and the like. Illustrative examples of photoinitiators are 1-hydroxycyclohexyl phenyl ketone, 2-hydroxy-2-methyl-1-phenyl-propan-1-one, bis(2,6-dimethoxybenzoyl)-2,4-4-trimethylpentyl phosphine oxide (DMBAPO), bis(2,4,6-trimethylbenzoyl)-phenyl phosphineoxide (Irgacure 819), 2,4,6-trimethylbenzyldiphenyl phosphine oxide and 2,4,6-trimethylbenzoyl diphenylphosphine oxide, benzoin methyl ester and a combination of camphorquinone and ethyl 4-(N,N-dimethylamino)benzoate. Commercially available visible light initiator systems include Irgacure 819, Irgacure 1700, Irgacure 1800, Irgacure 1850 (all from Ciba Specialty Chemicals) and Lucirin TPO initiator (available from BASF). Commercially available UV photoinitiators include Darocur 1173 and Darocur 2959 (Ciba Specialty Chemicals). These and other photoinitiators which may be used are disclosed in Volume III, Photoinitiators for Free Radical Cationic & Anionic Photopolymerization, 2^{nd} Edition by J. V. Crivello & K. Dietliker; edited by G. Bradley; John Wiley and Sons; New York; 1998, which is incorporated herein by reference.

The ligand monomers or their homopolymers, are mixed with the lens forming components in a diluent, prior to polymerization in an amount based on the weight percent of the initial monomer mix, including a suitable diluent if said diluent is used in the preparation of the polymer. The weight percentage of the ligand monomers can vary with the lens formulation. The maximum percentage of ligand monomers is the percentage that does not compromise the physical properties of the resulting contact lens, such as, but not limited to, modulus of the resulting lens. The minimum percentage of ligand monomers is an amount that allows the incorporation of a sufficient amount of silver into a lens to provide the desired antimicrobial effect. Preferably, about 0.01 to about 20.0 weight percent (based upon the total weight of lens forming components and ligand monomer) of ligand monomers is added, to a contact lens formulation, more preferably, about 0.01 to about 3 weight percent, and in some embodiments as little as 100 ppm to about 2000 ppm may be added.

Ligand monomers are added to the soft contact lens formulations described in U.S. Pat. No. 5,710,302, WO 9421698, EP 406161, JP 2000016905, U.S. Pat. No. 5,998,498, WO03/022322, WO03/022321, 5,760,100, 5,260,000 and U.S. Pat. No. 6,087,415. In addition, ligand monomers may be added to the formulations of commercial soft contact lenses. Examples of commercially available soft contact lenses formulations include but are not limited to, the formulations of etafilcon A, genfilcon A, lenefilcon A, polymacon, acquafilcon A, balafilcon A, senofilcon A, galyfilcon A and lotrafilcon A. The preferable contact lens formulations are etafilcon A, balafilcon A, lotrafilcon A, senofilcon A, galyfilcon A and silicone hydrogels, as prepared in U.S. Pat. No. 5,760,100; U.S. Pat. No. 5,776,999; U.S. Pat. No. 5,849,811; U.S. Pat. No. 5,789,461; U.S. Pat. No. 5,998,498, WO03/022321, WO03/022322 and 10/236,762, and U.S. Pat. No. 6,087,415.

Lenses prepared from the aforementioned formulations and the ligand monomers may be coated with a number of agents that are used to coat lenses. For example, the procedures, compositions, and methods of U.S. Pat. Nos. 3,854,982; 3,916,033; 4,920,184; and 5,002,794; 5,712,327; and 6,087,415 as well as WO 0127662, WO03/011551, may be used and these patents are hereby incorporated by reference for those procedures, compositions, and methods. In addition to the cited coating patents, there are other methods of treating a lens once it is formed. The lenses of this invention may be treated by these methods and the following publications which illustrate these methods are hereby incorporated by reference in their entirety, U.S. Pat. No. 5,453,467; U.S. Pat. No. 5,422,402; WO 9300391; U.S. Pat. No. 4,973,493; and U.S. Pat. No. 5,350,800.

Hard contact lenses are made from polymers that include but are not limited to polymers of poly(methyl)methacrylate, silicon acrylates, fluoroacrylates, fluoroethers, polyacetylenes, and polyimides, where the preparation of representative examples may be found in U.S. Pat. No. 4,330,383. Intraocular lenses of the invention can be formed using known materials. For example, the lenses may be made from a rigid material including, without limitation, polymethyl methacrylate, polystyrene, polycarbonate, or the like, and combinations thereof. Additionally, flexible materials may be used including, without limitation, hydrogels, silicone materials, acrylic materials, fluorocarbon materials and the like, or combinations thereof. Typical intraocular lenses are described in WO 0026698; WO 0022460; WO 9929750; WO 9927978; WO 0022459. The ligand monomers may be added to hard contact lens formulations and intraocular lens formulations in the same manner and at the same percentage as described above for soft contact lenses. All of the references mentioned in this application are hereby incorporated by reference in their entirety.

As used herein, the term “silver” refers to silver ions that are incorporated into a lens. While not wanting to be bound as to the oxidation state of the silver (Ag¹⁺ or Ag²⁺), that is incorporated into the lens, silver may be added to the lens by washing the cured and hydrated lens in a silver solution such as silver nitrate in deionized water (“DI”). Other sources of silver include but are not limited to silver acetate, silver citrate, silver iodide, silver lactate, silver picrate, and silver sulfate. The concentration of silver in these solutions can vary from the concentration required to add a known quantity of silver to a lens to a saturated silver solution. In order to calculate the concentration of the silver solution needed, the following calculation is used: the concentration of silver solution is equal to the desired amount of silver per lens, multiplied by the dry weight of the lens divided by the total volume of treating solution.
silver solution concentration (μg/mL)=[desired silver in lens (μg/g)×average dry lens weight (g)]/total volume of treating solution (mL)
For example, if one requires a lens containing 40 μg/g of silver, the dry weight of the lens is 0.02 g, and the vessel used to treat said lens has a volume of 3 mL, the required silver concentration would be 0.27 μg/mL.

It has been found that the ratio of the weight % silver to the weight % ligand in the lens should be greater than about 0.6 and preferably greater than about 0.8. When ratios of the present invention are used, log reductions in microbial adhesion of at least about 0.4 logs (cfu/lens) and preferably greater than about 1 log (cfu/lens) may be achieved.

Silver solutions containing anywhere from about 0.10 μg/mL to 0.3 grams/mL may be used depending upon the concentration of the ligand monomer used to prepare the lenses of the invention. Aside from deionized water, other liquid media can be used such as water, aqueous buffered solutions and organic solutions such as polyethers or alcohols. Typically, the lens is washed in the silver solution for about 60 minutes, though the time may vary from about 1 minute to about 2 hours and at temperatures ranging from about 5° C. to about 130° C. After the silver treatment the lenses are washed with several portions of water to obtain a lens where silver ions are releasably bound to the polymer via the ligand. The amount of silver that is incorporated into the lenses ranges from about 0.006 weight % (60 ppm) to about 10 weight % (100,000 ppm), where any lens containing at least about 60 ppm has the desired antimicrobial properties. The preferred amount of silver that is incorporated into the lens is about 60 ppm to about 4,000 ppm, more preferably, 60 ppm to about 2,000 ppm, even more preferably about 60 ppm to about 1,000 ppm.

The term “antimicrobial” refers to a lens that exhibit one or more of the following properties—the inhibition of the adhesion of bacteria or other microbes to the lenses, the inhibition of the growth of bacteria or other microbes on the lenses, and the killing of bacteria or other microbes on the surface of the lenses or in a radius extending from the lenses (hereinafter adhesion of bacteria or other microbes to the lenses, the growth of bacteria or other microbes to the lenses and the presence of bacterial or other microbes on the surface of lenses is collectively referred to as “microbial production”). The lenses of the invention inhibit the microbial production by at least 0.4 log reduction (≧60% inhibition). Preferably, the lenses of the invention exhibit at least a 1-log reduction (≧90% inhibition) of viable bacteria or other microbes, bacteria or other microbes. Such bacteria or other microbes include but are not limited to those organisms found in the eye, particularly Pseudomonas aeruginosa, Acanthamoeba species, Staphyloccus aureus, E. coli, Staphyloccus epidermidis, and Serratia marcesens. Preferably, said antimicrobial lens is a clear lens, that has clarity comparable to currently available commercial lenses such as but not limited to, etafilcon A, genfilcon A, lenefilcon A, polymacon, acquafilcon A, balafilcon A, galyfilcon, senofilcon and lotrafilcon A.

The advantages of the antimicrobial lenses of the invention are many. For example, other antimicrobial lenses that incorporate silver usually contain silver coordinated to some inorganic particulate matter. Often that particulate matter is visible to the naked or magnified eye, and it can affect the visual acuity of the user. However, the lenses of the invention do not have this problem. The ligand monomers are generally soluble with all of the other components of the antimicrobial lenses. Therefore when the lenses are produced they do not have substantial particulate matter due to their antimicrobial components. The antimicrobial lenses of the invention have comparable clarity to commercial lenses such as etafilicon A, genfilcon A, lenefilcon A, polymacon, acquafilcon A, balafilcon A, galyfilcon, senofilcon and lotrafilcon A.

Further, the invention includes a method of producing an antimicrobial lens comprising, silver and a polymer comprising at least one ligand monomer wherein
the method comprises, consists essentially of, or consists of the steps of
(a) preparing a lens comprising at least one ligand monomer, and
(b) treating said lens with a silver solution in an amount sufficient to
provide a silver to ligand monomer ratio of at least about 0.6.
   The terms lens, antimicrobial, ligand monomer and silver all have their aforementioned meanings and preferred ranges. The term, “silver solution” refers to any liquid medium containing silver. The liquid medium includes but is not limited to water, deionized water, aqueous buffered solutions, alcohols, polyols, and glycols, where the preferred medium is deionized water. The silver of the solution is typically a silver salt such as silver nitrate, silver acetate, silver citrate, silver iodide, silver lactate, silver picrate, and silver sulfate. The concentration of silver in these solutions can vary from the concentration required to add a known quantity of silver to a lens to a saturated silver solution. The concentration of the silver solution needed, may be calculated as described above.

Silver solutions containing anywhere from about 0.10 μg/mL to 0.3 grams/mL have been used to prepare the lenses of the invention. Typically, the lens is washed in the silver solution for about 60 minutes, though the time may vary from about 1 minute to about 2 hours and at temperatures ranging from about 5° C. to about 130° C. After the silver treatment the lenses are washed with several portions of water to obtain a lens where silver is incorporated into the polymer.

Still further, the invention includes a lens case comprising, consisting essentially of, or consisting of silver and a polymer of a ligand monomer as described above The term lens case refers to a container that is adapted to define a space in which to hold a lens when that lens is not in use. This term includes packaging for lenses, where packaging includes any unit in which a lens is stored after curing. Examples of this packaging include but are not limited to single use blister packs, multiple use storage cases and the like.

One such container is illustrated in FIG. 3 of U.S. Pat. No. 5,515,117. The ligand monomers can be incorporated in the lens container, the cover, or the lens basket, where they are preferably incorporated into the lens container or the lens basket.

Aside from the ligand monomer the container components may be made of a transparent, thermo-plastic polymeric material, such as polymethylmethacrylate, polyolefins, such as poly-ethylene, polypropylene, their copolymers and the like; polyesters, polyurethanes; acrylic polymers, such as polyacrylates and polymethacrylates; polycarbonates and the like and is made, or any combination thereof, e.g., molded, using conventional techniques as a single unit.

Silver may be incorporated into the lens container in the same manner that it is incorporated into the antimicrobial lenses of the invention. More specifically, the ligand monomer is combined with the formulation of the other components, molded, cured, and subsequently treated with a silver solution. Preferably, the ligand monomers are present in any or all of the lens case components at about 0.01 to about 10.0 weight percent (based on the initial monomer mix), more preferably about 0.01 to about 1.5 percent. Storing lenses in such an environment inhibits the growth of bacteria on said lenses and adverse effects that are caused by the proliferation of bacterial. Another example of such a lens case is the lens case can be found in U.S. Pat. No. 6,029,808 which is hereby incorporated by reference for the blister pack housing for a contact lens disclosed therein.

Yet still further, the invention includes a method of reducing the adverse effects associated with microbial production in the eye of a mammal, comprising, consisting essentially of, or consisting of providing an antimicrobial lens wherein said lens comprises silver and a polymer comprising at least one ligand monomer.

The phrase “adverse effects associated with microbial production” includes but is not limited to, ocular inflammation, contact lens related peripheral ulcers, contact lens associated red eye, infiltrative keratitis, and microbial keratitis.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

### EXAMPLES

The following abbreviations were used in the examples
PVP=polyvinylpyrrolidinone;
MAA=methacrylic acid;
PAA=poly(acrylic acid)
ATU=allylthiourea;
Cell/prot=(Acrylamidomethyl)cellulose acetate propionate
3M3P=3-methyl-3-propanol
D3O=3,7-dimethyl-3-octanol
TAA=t-amyl alcohol
BAGE=glycerin esterified with boric acid
DI=deionized water;
PBS=phosphate-buffered saline, pH 7.4±0.2;
TPBS=Phosphate-buffered saline with 0.05% Tween™ 80, pH 7.4±0.2;
TSA=sterile tryptic soy agar;
TSB=sterile tryptic soy broth;
60% IPA=isopropyl alcohol, 60% v/v DI;
70% IPA=isopropyl alcohol, 70% v/v Dl;
10% IPA=isopropyl alcohol, 10% v/v DI;
MVD=modified vortex device;
TBACB=tetrabutyl ammonium-m-chlorobenzoate
TMI=dimethyl meta-isopropenyl benzyl isocyanate
MMA=methyl methacrylate
HEMA=hydroxyethyl methacrylate
mPDMS=mono-methacryloxypropyl terminated polydimethylsiloxane MW=800-1000
DMA=N,N-dimethylacrylamide
Blue HEMA=the reaction product of reactive blue number 4 and HEMA as described in Example 4 of U.S. Pat. No. 5,944,853
DAROCUR 1173=2-hydroxy-2-methyl-1-phenyl-propan-1-one
EGDMA=ethyleneglycol dimethacrylate
TMPTMA=trimethyloyl propane trimethacrylate
TEGDMA=tetraethyleneglycol dimethacrylate
Norbloc=2-(2′-hydroxy-5-methacrylyloxyethylphenyl)-2H-benzotriazole
CGI 1850=1:1 (w/w) blend of 1-hydroxycyclohexyl phenyl ketone and bis(2,6-dimethyoxybenzoyl)-2,4-4-trimethylpentyl phosphine oxide
w/w=weight/total weight
w/v=weight/total volume
v/v=volume/total volume
pHEMA=poly(hydroxyethyl)methacrylate coating as described in Example 14 of U.S. Ser. No. 09/921,192, “Methods for Coating Articles by Mold Transfer”

The contact lenses of the invention were evaluated for antibacterial efficacy using the following biological assay: A culture of Pseudomonas aeruginosa, ATCC# 15442 (American Type Culture Collection, Rockville, Md.), was grown overnight in a tryptic soy medium. The culture was washed three times in phosphate buffered saline (PBS, pH=7.4+/−0.2) and the bacterial pellet was resuspended in 10 ml of PBS. The bacterial inoculum was prepared to result in a final concentration of approximately 1×10⁶ colony forming units/mL (cfu/mL). Three contact lenses were rinsed in three changes of 30 milliliters of phosphate buffered saline (PBS, pH=7.4+/−0.2) to remove residual solutions. Each rinsed lens was placed with 2 mL of the bacterial inoculum into a sterile glass vial, which was then rotated in a shaker-incubator (100 rpm) for two hours at 37+/−2° C. Each lens was removed from the glass vial, rinsed five times in three changes of PBS to remove loosely bound cells, placed into individual wells of a 24-well microtiter plate containing 1 mL PBS, and rotated in a shaker-incubator for an additional 22 hours at 37+/−2° C. Each lens was again rinsed five times in three changes of PBS to remove loosely bound cells, placed into 10 mL of PBS containing 0.05% (w/v) Tween™ 80, and vortexed at 2000 rpm for 3 minutes, employing centrifugal force to disrupt adhesion of the remaining bacteria to the lens. The resulting supernatant was enumerated for viable bacteria and the results of detectable viable bacteria attached to 3 lenses were averaged and this data is presented as the log reduction of the innoculum, as compared to control (lenses made from the Table 1 formulation without added silver).

Silver content was determined by Instrumental Neutron Activation Analysis “INM”. INAA is a qualitative and quantitative elemental analysis method based on the artificial induction of specific radionuclides by irradiation with neutrons in a nuclear reactor. Five lenses are placed individually in 20 ml polypropylene scintillation vials and dried in a vacuum oven at approximately 60° C. for a minimum of 4 hours. The lenses were individually weighed and placed in irradiation vials and analyzed. Irradiation of the sample is followed by the quantitative measurement of the characteristic gamma rays emitted by the decaying radionuclides. The gamma rays detected at a particular energy are indicative of a particular radionuclide's presence, allowing for a high degree of specificity. Becker, D. A.; Greenberg, R. R.; Stone, S. F. J. Radioanal. Nucl. Chem. 1992, 160(1), 41-53; Becker, D. A.; Anderson, D. L.; Lindstrom, R. M.; Greenberg, R. R.; Garrity, K. M.; Mackey, E. A. J. Radioanal. Nucl. Chem. 1994, 179(1), 149-54. The INAA procedure used to quantify silver content in contact lens material uses the following two nuclear reactions:

1. In the activation reaction, ¹¹⁰Ag is produced from stable ¹⁰⁹Ag (isotopic abundance=48.16%) after capture of a radioactive neutron produced in a nuclear reactor.

2. In the decay reaction, ¹¹⁰Ag (τ^{1/2}=24.6 seconds) decays primarily by negatron emission proportional to initial concentration with an energy characteristic to this radio-nuclide (657.8 keV).

The gamma-ray emission specific to the decay of ¹¹⁰Ag from irradiated. standards and samples are measured by gamma-ray spectroscopy, a well-established pulse-height analysis technique, yielding a measure of the concentration of the analyte.

Th weight % ATU in the lenses is measured using HPLC. Three lenses weighed into a 20 ml glass scintillation vial and extracted with methanol. The extract is analyzed by HPLC using the following conditions:
Column: Prodigy ODS3 150+4.6 mm, 5 um particle diameter
mobile phase: 5% methanol 95% water
detector wavelength: 210 nm
injection volume: 10 ul
flow rate: 1 ml/min

The amount of ATU in the extract is quantified by comparison of ATU peak area against external standards. The amount of ATU incorporated (i.e. co-polymerized) into the polymer is calculated by subtracting this value from the nominal concentration.

### Example 1

To a dry container housed in a dry box under nitrogen at ambient temperature was added 30.0 g (0.277 mol) of bis(dimethylamino)methylsilane, a solution of 13.75 mL of a 1 M solution of TBACB (386.0 g TBACB in 1000 mL dry THF), 61.39 g (0.578 mol) of p-xylene, 154.28 g (1.541 mol)methyl methacrylate (1.4 equivalents relative to initiator), 1892.13 (9.352 mol) 2-(trimethylsiloxy)ethyl methacrylate (8.5 equivalents relative to initiator) and 4399.78 g (61.01 mol) of THF. To a dry, three-necked, round-bottomed flask equipped with a thermocouple and condenser, all connected to a nitrogen source, was charged the above mixture prepared in the dry box.

The reaction mixture was cooled to 15° C. while stirring and purging with nitrogen. After the solution reached 15° C., 191.75 g (1.100 mol) of 1-trimethylsiloxy-1-methoxy-2-methylpropene (1 equivalent) was injected into the reaction vessel. The reaction was allowed to exotherm to approximately 62° C. and then 30 mL of a 0.40 M solution of 154.4 g TBACB in 11 mL of dry THF was metered in throughout the remainder of the reaction. After the temperature of reaction reached 30° C. and the metering began, a solution of 467.56 g (2.311 mol) 2-(trimethylsiloxy)ethyl methacrylate (2.1 equivalents relative to the initiator), 3636.6. g (3.463 mol) n-butyl monomethacryloxypropyl-polydimethylsiloxane (3.2 equivalents relative to the initiator), 3673.84 g (8.689 mol), TRIS (7.9 equivalents relative to the initiator) and 20.0 g bis(dimethylamino)methylsilane was added.

The mixture was allowed to exotherm to approximately 38-42° C. and then allowed to cool to 30° C. At that time, a solution of 10.0 g (0.076 mol) bis(dimethylamino)methylsilane, 154.26 g (1.541 mol)methyl methacrylate (1.4 equivalents relative to the initiator) and 1892.13 g (9.352 mol) 2-trimethylsiloxy)ethyl methacrylate (8.5 equivalents relative to the initiator) was added and the mixture again allowed to exotherm to approximately 40° C. The reaction temperature dropped to approximately 30° C. and 2 gallons of THF were added to decrease the viscosity. A solution of 439.69 g water, 740.6 g methanol and 8.8 g (0.068 mol) dichloroacetic acid was added and the mixture refluxed for 4.5 hours to de-block the protecting groups on the HEMA. Volatiles were then removed and toluene added to aid in removal of the water until a vapor temperature of 110° C. was reached.

The reaction flask was maintained at approximately 110° C. and a solution of 443 g (2.201 mol) TMI and 5.7 g (0.010 mol) dibutyltin dilaurate were added. The mixture was reacted until the isocyanate peak was gone by IR. The toluene was evaporated under reduced pressure to yield an off-white, anhydrous, waxy reactive monomer. The macromer was placed into acetone at a weight basis of approximately 2:1 acetone to macromer. After 24 hrs, water was added to precipitate out the macromer and the macromer was filtered and dried using a vacuum oven between 45 and 60° C. for 20-30 hrs.

### Examples 2-4

Reactive monomer mixes were formed by dissolving the components, in the percentages listed in Table 1 and ATU in the amounts listed in Table 2, with D30 in an 80:20 weight % mixture as follows: the components listed in Table 1 and ATU were mixed with D30 in an Erlenmeyer flask, sonicated at approximately 45° C. until all components are dissolved and were subsequently loaded into an eight cavity lens mold of the type described in U.S. Pat. No. 4,640,489 and cured for 30 minutes at 55° C. Polymerization occurred under a nitrogen purge and was photoinitiated with 5 mW cm⁻² visible light generated with a Philips TL 20W/03T fluorescent bulb. After curing, the molds were opened, and the lenses were released in a 60% PA/water, then leached in IPA/DI to remove any residual monomers and diluent. Finally the lenses were equilibrated in either physiological borate-buffered saline or de-ionized water.

TABLE 1

| Component | Weight % |
|---|---|
| Macromer | 17.98 |
| TRIS | 14 |
| DMA | 26 |
| MPDMS | 28 |
| Norbloc | 2 |
| CGI 1850 | 1 |
| TEGDMA | 1 |
| HEMA | 5 |
| Blue HEMA | 0.02 |
| PVP | 5 |

### Examples 5-7

A stock solution of silver nitrate in DI water was prepared (1.0157 g AgNO_{3/100} ml water). The AgNO₃ solution was diluted 1:100 in DI water. The lenses prepared in Examples 2-4 above were placed in glass vials with 3 ml special packing solution (“SPS” which contains the following in deionized H₂O: 0.18 weight % sodium borate [1330-43-4], Mallinckrodt and 0.91 weight % boric acid [10043-35-3], Mallinckrodt) per lens. Silver nitrate was added to each vial in a volume calculated to provide the desired silver to ATU ratio. The vials containing the lenses were autoclaved for 2 hours at 121° C. The treated lenses were removed from the silver solution and placed into distilled water (300 mL). The lenses were either rolled or stirred in distilled water for about 30 minutes. This water washing procedure was repeated three (3) more times. The resulting lenses were stored in saline solution and tested to determine their antimicrobial potential. The results of the bacterial adhesion assay are presented in Table 2, below. In addition, the lenses were analyzed by instrumental neutron activation analysis, to determine the amount of silver that was incorporated in the lenses. This data is presented in Table 2.

| | | | | Log Redxn | |
|---|---|---|---|---|---|
| Ex | Ag | ATU target | ATU | adhesion | |
| # | (ppm) | (wt %) | (wt %) | (cfu/lens) | [Ag]/[ATU] |
| 5 | 967 ± 28 | 0.1 | 0.124 | 1.04 ± 0.07 | 0.84 |
| 6 | 974 ± 53 | 0.2 | 0.179 | 0.41 ± 0.35 | 0.58 |
| 7 | 953 ± 22 | 0.5 | 0.292 | 0.16 ± 0.36 | 0.35 |

1. An antimicrobial lens comprising silver and a polymer formed from a reaction mixture comprising at least one ligand monomer of Formula I 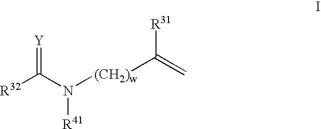
wherein
w is 0-1;
Y is oxygen or sulfur;
R³¹ is hydrogen or C₁₋₆alkyl;
R³² is selected from the group consisting of hydroxyl, amino, sulfonic acid, phosphonic acid, carboxylic acid, thioC₁₋₆alkylcarbonyl, thioC₁₋₆alkylaminocarbonyl, —C(O)NH—(CH₂)_{d} —R³³, —O—R³³, —NH—R³³, —S—(CH₂)_{d} —R³³, —(CH₂)_{d} —R³³, C₁₋₆alkyldisulfide, phenyldisulfide, urea, C₁₋₆alkylurea, phenylurea, thiourea, C₁₋₆alkylthiourea, phenylthiourea, C₁₋₆alkylamine, phenylamine, substituted C₁₋₆alkyldisulfide, substituted phenyldisulfide, substituted phenylurea, substituted C₁₋₆alkylamine, substituted phenylamine, substituted phenylthiourea, substituted C₁₋₆alkylurea or substituted C₁₋₆alkylthiourea wherein the substitutents are selected from the group consisting of C₁₋₆alkyl, haloC₁₋₆alkyl, halogen, hydroxyl, carboxylic acid, sulfonic acid, phosphonic acid, amine, amidine, acetamide, and nitrile
where
d is 0-8;
R³³ is thioC₁₋₆alkylcarbonyl, C₁₋₆alkyl, substituted C₁₋₆alkyl
where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea or substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR³⁴R³⁵)_{q}—(CHR³⁶)ₘ—SO₃H
where R³⁴, R³⁵, and R³⁶ are independently selected from the group consisting of hydrogen, halogen, hydroxyl, and C₁₋₆alkyl,
q is 1-6, and m is 0-6;
—(CH₂)ₙ—S—S—(CH₂)ₓNH—C(O)CR³⁷CH₂,
where R³⁷ is hydrogen or C₁₋₆alkyl,
n is 1-6, and x is 1-6;
—(CR³⁸R³⁹)ₜ—(CHR⁴⁰)ᵤ—P(O)(OH)₂
where R³⁸, R3⁹, and R⁴⁰ are independently selected from the group consisting of hydrogen, halogen, hydroxyl, and C₁₋₆alkyl,
t is 1-6, and
u is 0-6;
phenyl, benzyl, pyridinyl, pyrimidinyl, pyrazinyl, benzimidazolyl, benzothiazolyl, benzotriazolyl, naphthaloyl, quinolinyl, indolyl, thiadiazolyl, triazolyl, 4-methylpiperidin-1-yl, 4-methylpiperazin-1-yl, substituted phenyl, substituted benzyl, substituted pyridinyl, substituted pyrimidinyl, substituted pyrazinyl, substituted benzimidazolyl, substituted benzothiazolyl, substituted benzotriazolyl, substituted naphthaloyl, substituted quinolinyl, substituted indolyl, substituted thiadiazolyl, substituted triazolyl, substituted 4-methylpiperidin-1-yl, or substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperid inyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl)phosphonyl, N-(amino-4-methylpiperazinyl)phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
R⁴¹ is selected from the group consisting of hydrogen, C₁₋₆alkyl, phenyl, C₁₋₆alkylcarbonyl, phenylcarbonyl, substituted C₁₋₆alkyl, substituted phenyl, substituted C₁₋₆alkylcarbonyl and substituted phenylcarbonyl,
wherein
the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile
wherein the silver is releasably bound to the ligand, and the silver is present in the lens in an amount, expressed as a ratio of silver to ligand monomer of at least about 0.6.

2. The antimicrobial lens of claim 1 wherein,
w is 0-1;
R³¹ is hydrogen;
R³² is selected from the group consisting of amine, C₁₋₃alkylamine, phenylamine, substituted phenylamine, thioC₁₋₃alkylcarbonyl; and
R⁴¹ is hydrogen

3. The antimicrobial lens of claim 1 wherein the lens is a soft contact lens.

4. The antimicrobial lens of claim 1 wherein the monomer of Formula I is present at about 0.01 to about 1.5 weight percent.

5. The antimicrobial lens of claim 1 wherein the ligand monomer is present at about 0.01 to about 0.8 weight percent.

6. The antimicrobial lens of claim 1 wherein the ligand monomer is present at about 0.01 to about 0.3 weight percent.

7. The antimicrobial lens of claim 1 wherein the ligand monomer is present at about 0.01 to about 0.2 weight percent.

8. The antimicrobial lens of claim 1 wherein the ratio of silver to ligand monomer is at least about 0.8.

9. The antimicrobial lens of claim 1 wherein the lens is a silicone hydrogel.

10. The antimicrobial lens of claim 1 wherein, the lens is etafilcon A, balafilcon, A, acquafilcon A, lenefilcon A, galyfilcon, senofilcon or lotrafilcon A.

11. The antimicrobial lens of claim 1 wherein,
R¹, R⁴, R⁵, R⁶, R⁸, R⁹ and R¹⁰ are independently hydrogen or methyl;
R² is NH—R³;
R³ is —(CR⁴, R⁵)_{q}—(CHR⁶)ₘ—SO₃H, —(CR⁸R⁹)ₜ—(CHR¹⁰)ᵤ—P(O)(OH)₂ or —(CH₂)ₙ—S—S—(CH₂)ₙNH—C(O)CHR⁷CH₂;
q is 1-2; m is 1-2; R⁷ is hydrogen; t is 1; u is 1-2; n is 2-3; and
x is 2-3.

12. The antimicrobial lens of claim 1 wherein the monomer of Formula I is selected from the group consisting of 1-allyl-2 thiourea and the following monomers

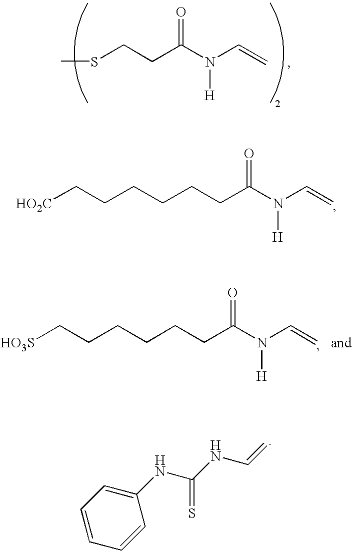

13. The antimicrobial lens of claim 1 wherein silver is present at about 60 ppm to about 4,000 ppm.

14. The antimicrobial lens of claim 1 wherein silver is present at about 60 ppm to about 2,000 ppm.

15. The antimicrobial lens of claim 1 wherein silver is present at about 60 ppm to about 1,000 ppm.

16. The antimicrobial lens of claim 1 wherein the lens is a silicone hydrogel and the ligand monomer is 1-allyl-2-thiourea.

17. The antimicrobial lens of claim 16 wherein silver is present at about 60 ppm to about 4000 ppm and the ligand monomer is present at about 0.01 to about 1.5 weight percent.

18. The antimicrobial lens of claim 1 wherein the lens is etafilcon A, balafilcon, A, acquafilcon A, lenefilcon, galyfilcon, senofilcon or lotrafilcon A and the ligand monomer is 1-allyl-2-thiourea.

19. The antimicrobial lens of claim 18 wherein silver is present at about 60 ppm to about 2000 ppm and the ligand monomer is present at about 0.01 to about 1.5 weight percent.

20. The antimicrobial lens of claim 19 wherein the lens is etafilcon A or acquafilcon A.

21. The lens of claim 20 wherein silver is present at about 60 ppm to about 1000 ppm.

22. A method of producing an antimicrobial lens comprising, silver and a polymer comprising at least one ligand monomer of Formula I 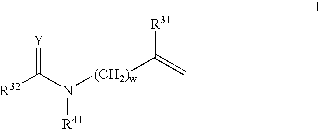
wherein
w is 0-1;
Y is oxygen or sulfur;
R³¹ is hydrogen or C₁₋₆alkyl;
R³² is selected from the group consisting of hydroxyl, amino, sulfonic acid, phosphonic acid, carboxylic acid, thioC₁₋₆alkylcarbonyl, thioC₁₋₆alkylaminocarbonyl, —C(O)NH—(CH₂)_{d} —R³³, —O—R³³, —NH—R³³, —S—(CH₂)_{d} —R³³, —(CH₂)_{d} —R³³, C₁₋₆alkyldisulfide, phenyldisulfide, urea, C₁₋₆alkylurea, phenylurea, thiourea, C₁₋₆alkylthiourea, phenylthiourea, C₁₋₆alkylamine, phenylamine, substituted C₁₋₆alkyldisulfide, substituted phenyldisulfide, substituted phenylurea, substituted C₁₋₆alkylamine, substituted phenylamine, substituted phenylthiourea, substituted C₁₋₆alkylurea or substituted C₁₋₆alkylthiourea wherein the substitutents are selected from the group consisting of C₁₋₆alkyl, haloC₁₋₆alkyl, halogen, hydroxyl, carboxylic acid, sulfonic acid, phosphonic acid, amine, amidine, acetamide, and nitrile
where
d is 0-8;
R³³ is thioC₁₋₆alkylcarbonyl, C₁₋₆alkyl, substituted C₁₋₆alkyl
where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea or substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR³⁴R³⁵)_{q}—(CHR³⁶)ₘ—SO₃H
where R³⁴, R³⁵, and R³⁶ are independently selected from the group consisting of hydrogen, halogen, hydroxyl, and C₁₋₆alkyl,
q is 1-6, and m is 0-6;
—(CH₂)ₙ—S—S—(CH₂)ₓNH—C(O)CR³⁷CH₂,
where R³⁷ is hydrogen or C₁₋₆alkyl,
n is 1-6, and x is 1-6;
—(CR³⁸R³⁹)ₜ—(CHR⁴⁰)—P(O)(OH)₂
where R³⁸, R3⁹, and R⁴⁰ are independently selected from the group consisting of hydrogen, halogen, hydroxyl, and C₁₋₆alkyl,
t is 1-6, and
u is 0-6;
phenyl, benzyl, pyridinyl, pyrimidinyl, pyrazinyl, benzimidazolyl, benzothiazolyl, benzotriazolyl, naphthaloyl, quinolinyl, indolyl, thiadiazolyl, triazolyl, 4-methylpiperidin-1-yl, 4-methylpiperazin-1-yl, substituted phenyl, substituted benzyl, substituted pyridinyl, substituted pyrimidinyl, substituted pyrazinyl, substituted benzimidazolyl, substituted benzothiazolyl, substituted benzotriazolyl, substituted naphthaloyl, substituted quinolinyl, substituted indolyl, substituted thiadiazolyl, substituted triazolyl, substituted 4-methylpiperidin-1-yl, or substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl)phosphonyl, N-(amino-4-methylpiperazinyl)phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
R⁴¹ is selected from the group consisting of hydrogen, C₁₋₆alkyl, phenyl, C₁₋₆alkylcarbonyl, phenylcarbonyl, substituted C₁₋₆alkyl, substituted phenyl, substituted C₁₋₆alkylcarbonyl and substituted phenylcarbonyl,
wherein
the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile
where the method comprises the steps of
(a) preparing a lens comprising at least one ligand monomer and
(b) treating the lens with a silver solution of a concentration to provide the lens with a silver to ligand monomer ratio of at least about 0.6.

23. The method of claim 22 wherein the silver solution is aqueous silver nitrate having a concentration of about 0.1 μg/mL to about 0.3 g/mL.

24. The method of claim 22 wherein, the treating step comprises soaking the lens in the silver solution.

25. The method of claim 24 wherein, the lens is soaked in the silver solution for about 2 minutes to about 2 hours.

26. The method of claim 22 wherein, the treating step comprises storing the lens in a silver solution for about 20 minutes to about 5 years.

27. The method of claim 22 wherein the ratio of silver to ligand monomer is at least about 0.8.

28. The lens of claim 1 wherein said lens displays at least about a 0.4 log reduction in microbial activity.

29. The lens of claim 1 wherein said lens displays at least about a 1 log reduction in microbial activity.

30. A lens case comprising silver and a polymer comprising at least one ligand monomer of Formula I
of Formula I 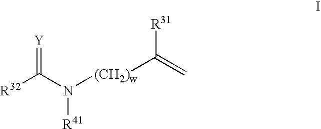
wherein
w is 0-1;
Y is oxygen or sulfur;
R³¹ is hydrogen or C₁₋₆alkyl;
R³² is selected from the group consisting of hydroxyl, amino, sulfonic acid, phosphonic acid, carboxylic acid, thioC₁₋₆alkylcarbonyl, thioC₁₋₆alkylaminocarbonyl, —C(O)N H—(CH₂)_{d} —R³³, —O—R³³, —NH—R³³, —S—(CH₂)_{d} —R³³, —(CH₂)_{d} —R³³, C₁₋₆alkyldisulfide, phenyldisulfide, urea, C₁₋₆alkylurea, phenylurea, thiourea, C₁₋₆alkylthiourea, phenylthiourea, C₁₋₆alkylamine, phenylamine, substituted C₁₋₆alkyldisulfide, substituted phenyldisulfide, substituted phenylurea, substituted C₁₋₆alkylamine, substituted phenylamine, substituted phenylthiourea, substituted C₁₋₆alkylurea or substituted C₁₋₆alkylthiourea wherein the substitutents are selected from the group consisting of C₁₋₆alkyl, haloC₁₋₆alkyl, halogen, hydroxyl, carboxylic acid, sulfonic acid, phosphonic acid, amine, amidine, acetamide, and nitrile
where
d is 0-8;
R³³ is thioC₁₋₆alkylcarbonyl, C₁₋₆alkyl, substituted C₁₋₆alkyl
where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea or substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR³⁴R³⁵)_{q}—(CHR³⁶)ₘ—SO₃H
where R³⁴, R³⁵, and R³⁶ are independently selected from the group consisting of hydrogen, halogen, hydroxyl, and C₁₋₆alkyl,
q is 1-6, and m is 0-6;
—(CH₂)ₙ—S—S—(CH₂)ₙNH—C(O)CR³⁷CH₂,
where R³⁷ is hydrogen or C₁₋₆alkyl,
n is 1-6, and x is 1-6;
—(CR³⁸R³⁹)ₜ—(CHR⁴⁰)ᵤ—P(O)(OH)₂
where R³⁸, R3⁹, and R⁴⁰ are independently selected from the group consisting of hydrogen, halogen, hydroxyl, and C₁₋₆alkyl,
t is 1-6, and
u is 0-6;
phenyl, benzyl, pyridinyl, pyrimidinyl, pyrazinyl, benzimidazolyl, benzothiazolyl, benzotriazolyl, naphthaloyl, quinolinyl, indolyl, thiadiazolyl, triazolyl, 4-methylpiperidin-1-yl, 4-methylpiperazin-1-yl, substituted phenyl, substituted benzyl, substituted pyridinyl, substituted pyrimidinyl, substituted pyrazinyl, substituted benzimidazolyl, substituted benzothiazolyl, substituted benzotriazolyl, substituted naphthaloyl, substituted quinolinyl, substituted indolyl, substituted thiadiazolyl, substituted triazolyl, substituted 4-methylpiperidin-1-yl, or substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl)phosphonyl, N-(amino-4-methylpiperazinyl)phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
R⁴¹ is selected from the group consisting of hydrogen, C₁₋₆alkyl, phenyl, C₁₋₆alkylcarbonyl, phenylcarbonyl, substituted C₁₋₆alkyl, substituted phenyl, substituted C₁₋₆alkylcarbonyl and substituted phenylcarbonyl,
wherein
the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile.
